(19) United States
(12) Patent Application Publication
(10) Pub. No.: US 2013/0323324 A1
(43) Pub. Date: Dec. 5, 2013

(54) VANADIUM AND VANADYL AMINO ACID COMPLEXES

(71) Applicant: Franco Cavaleri, Surrey (CA)

(72) Inventor: Franco Cavaleri, Surrey (CA)

(21) Appl. No.: 13/907,866

(22) Filed: Jun. 1, 2013

Related U.S. Application Data
(60) Provisional application No. 61/654,663, filed on Jun. 1, 2012.

(51) Int. Cl. A61K 47/48; A61K 33/24

(52) U.S. Cl. CPC A61K 47/481; A61K 33/24
USPC 424/646; 556/44; 514/492; 530/331; 514/21.9; 556/42

(57) ABSTRACT

Compositions that include vanadium or vanadyl chelated to an amino acid are disclosed, particularly vanadium or vanadyl chelated to creatine, arginine, citrulline, taurine, phenylalanine, glutamine, glutathione, leucine, or combinations thereof. In addition, compositions that included blended forms of such chelates and/or vanadyl sulfate and one or more unbound amino acids. Still further, the compositions disclosed may further include one or more additional proteins, amino acids, vitamins, minerals, or combinations thereof, which impart an additional nutritional and/or therapeutic benefit to the composition.

### CROSS-REFERENCE TO RELATED APPLICATIONS

This application claims priority to, and incorporates by reference, U.S. provisional patent application Ser. No. 61/654,663, filed on Jun. 1, 2012.

### FIELD OF THE INVENTION

The field of the present invention relates to certain vanadium and vanadyl amino acid chelates, blends of vanadium (or vanadyl) and certain amino acids, and various methods of using the foregoing compositions.

### BACKGROUND OF THE INVENTION

The use of vanadium salts to modulate insulin activity, as well as the influence of such vanadium salts on other aspects of human health, has been heavily researched since the early 1990s. These studies have provided a fairly broad and deep understanding of how vanadium can be used to impart a therapeutic response for the treatment of various ailments. However, a need continues to exist for new therapeutic and nutritional formulations that provide the benefits of vanadium, in combination with other agents that may operate in a synergistic manner with vanadium to provide a more enhanced response (and/or other beneficial attributes). More particularly, there is a continuing need for vanadium- and/or vanadyl-containing formulations which exhibit enhanced bioavailability, reduced toxicity, and other advantageous attributes.

### SUMMARY OF THE INVENTION

According to certain aspects of the present invention, compositions comprising vanadium or vanadyl chelated to an amino acid are provided. According to such aspects of the invention, the amino acid is preferably selected from the group consisting of creatine, arginine, citrulline, taurine, phenylalanine, glutamine, glutathione, and leucine. According to certain related aspects of the present invention, combinations of such chelates are provided, which include vanadium or vanadyl chelated to a first type of amino acid and vanadium or vanadyl chelated to a second type of amino acid (and, in some embodiments, such combinations may encompass 3, 4, 5 or more different types of such chelates, with each vanadium or vanadyl chelated to a different amino acid).

According to additional aspects of the present invention, the compositions may include vanadyl sulfate and one or more unbound amino acids selected from the group consisting of creatine, arginine, citrulline, taurine, phenylalanine, glutamine, glutathione, and leucine. Such blended forms of vanadyl sulfate and one or more of such unbound amino acids may be formulated with, or without, the above-mentioned vanadium or vanadyl chelates. According to certain related aspects of the present invention, compositions that include vanadium blended with an unbound amino acid—selected from the group consisting of creatine, arginine, citrulline, taurine, phenylalanine, glutamine, glutathione, and leucine—are provided.

According to yet further aspects of the present invention, the above-described compositions may further be blended with one or more additional proteins, vitamins, minerals, amino acids, or combinations thereof, which impart a nutritional and/or therapeutic benefit to the composition. In certain preferred embodiments, for example, the above-described compositions may further be blended with whey proteins (and/or other milk-derived proteins).

According to still further aspects of the present invention, the invention encompasses methods of using various forms of the above-described compositions. More particularly, for example, the invention encompasses administering (1) vanadium/vanadyl chelated to creatine to support anabolic activity in the building of lean muscle mass, strength and power, fast twitch and slow twitch myofibril power and strength; (2) vanadium/vanadyl chelated to arginine to induce vasodilation and promote peripheral blood flow to support healthy blood pressure, optimum growth hormone output, maximum protein synthesis for recovery, optimal workout results, and improved male erectile performance; (3) vanadium/vanadyl chelated to citrulline to induce vasodilation and promote peripheral blood flow to support healthy blood pressure, optimum growth hormone output, maximum protein synthesis for recovery, optimal workout results, and improved male erectile performance; (4) vanadium/vanadyl chelated to taurine to support insulin activity in the treatment of Type I and Type II diabetes and the optimization of insulin efficiency in the case of subclinical insulin resistance; (5) vanadium/vanadyl chelated to phenylalanine to enhance serotonin survival, improve emotional state and weight management, and optimize insulin efficiency to improve fat loss, lean body mass status and healthy weight management; (6) vanadium/vanadyl chelated to glutamine to support immune system efficiency and lean muscle anabolism; (7) vanadium/vanadyl chelated to glutathione to support immune system efficiency in the course of treatment of disease and disease management, for antimicrobial and anti-aging activity, and for the preservation of tissue structure and function in response to toxicity, poisons and general oxidation pressures; and (8) vanadium/vanadyl chelated to leucine to support insulin efficiency and anabolic activity in the building of lean muscle mass, strength and power, to support healthy bone mass and reduced body fat, to improve lean body mass to fat body mass proportions in the course of treating obesity, to prevent the effects of osteoporosis and premature aging, to enhance performance in sports, and to support recovery from disease (such as insulin resistance, inflammatory disease or injury such as burns and tissue tears).

The above-mentioned and additional features of the present invention are further illustrated in the Detailed Description contained herein.

### DETAILED DESCRIPTION OF THE INVENTION

The following will describe, in detail, several preferred embodiments of the present invention. These embodiments are provided by way of explanation only, and thus, should not unduly restrict the scope of the invention. In fact, those of ordinary skill in the art will appreciate upon reading the present specification and viewing the present drawings that the invention teaches many variations and modifications, and that numerous variations of the invention may be employed, used and made without departing from the scope and spirit of the invention.

According to certain preferred embodiments, the present invention encompasses certain vanadium- and vanadyl-containing formulations, which further include one or more amino acids. More particularly, according to certain preferred embodiments, the formulations of the present invention include vanadium and/or vanadyl chelated with an amino acid residue. As used herein, the terms “chelate,” “chelated to,” “chelates,” and similar terms refer to a chemical compound in the form of a heterocyclic ring that comprises a metal ion, in this case vanadium (V) or the vanadyl ion (VO²⁺), attached by coordinate bonds to at least two other components, in this case at least two amino acid residues.

According to certain preferred embodiments of the present invention, compositions comprising vanadium or vanadyl chelated to an amino acid are provided. According to such embodiments, the amino acid is selected from the group consisting of creatine, arginine, citrulline, taurine, phenylalanine, glutamine, glutathione, and leucine. In addition, the invention provides that combinations of such chelates are provided, which include vanadium or vanadyl chelated to a first type of amino acid and vanadium or vanadyl chelated to a second type of amino acid. Still further, in certain embodiments, such combinations may encompass 3, 4, 5 or more different types of such chelates, e.g., a mixture of vanadium or vanadyl chelated to any of 3, 4, 5 or more different types of amino acids (with such amino acids preferably selected from creatine, arginine, citrulline, taurine, phenylalanine, glutamine, glutathione, and leucine).

According to additional embodiments, the present invention provides that the compositions may include vanadyl sulfate (VOSO₄), along with one or more unbound amino acids selected from the group consisting of creatine, arginine, citrulline, taurine, phenylalanine, glutamine, glutathione, and leucine. That is, the invention further encompasses vanadyl sulfate blended with unbound forms of such amino acids (or a select number of such amino acids). According to certain related embodiments, the present invention further encompasses compositions that include vanadium blended with such unbound amino acids, which are preferably selected from the group consisting of creatine, arginine, citrulline, taurine, phenylalanine, glutamine, glutathione, and leucine. The foregoing blended forms of vanadyl sulfate (or vanadium) and one or more of such unbound amino acids may be formulated with, or without, the vanadium and/or vanadyl chelates described above.

According to yet further embodiments of the present invention, the above-described compositions may further be blended with one or more additional proteins, vitamins, minerals, amino acids, or combinations thereof, which impart additional nutritional and/or therapeutic benefits to the composition. Non-limiting examples of such proteins include whey proteins, and/or other proteins derived from milk. In addition, the formulations of the present invention may further comprise vitamins, minerals and/or herbs that have specific and desirable therapeutic or nutritional properties.

The chelates encompassed by the present invention may be produced by reacting a suitable vanadium- or vanadyl-containing salt with the desired amino acid. For example, in the case of the vanadium chelates described herein, vanadium oxysulfate (O₅SV) may be reacted with the desired amino acid (in the presence of, e.g., sodium hydroxide) to achieve the desired chelate, i.e., vanadium oxysulfate+2RCHNH₂COOH and 2NaOH=desired chelate (with the R group varying depending on the selected amino acid). Similarly, in the case of the vanadyl chelates described herein, vanadyl sulfate (VOSO₄) may be reacted with the desired amino acid (in the presence of, e.g., sodium hydroxide) to achieve the desired chelate, i.e., vanadyl sulfate+2RCHNH₂COOH and 2NaOH=desired chelate (with the R group varying depending on the selected amino acid). According to such embodiments, the desired amino acid is reacted with the divalent vanadyl ion (VO⁺²) and its divalent metal (where the chelation occurs), as illustrated below.

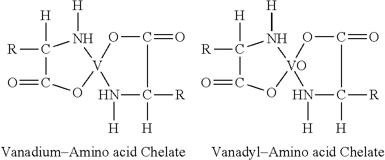

Accordingly, and as discussed herein, the present invention encompasses the chelation of the vanadium metal, as well as the vanadyl ion, thereby producing two separate but similar molecules.

The invention provides that the chelates of the present invention may be administered in any desired and effective manner, e.g., as pharmaceutical compositions or nutritional supplements for oral ingestion. More particularly, for example, pharmaceutically acceptable compositions or nutritional supplements of the invention comprise one or more of the chelates described herein with one or more acceptable carriers and, optionally, one or more other compounds, drugs, ingredients and/or materials. Regardless of the route of administration selected, the chelates may be formulated into acceptable dosage forms by conventional methods known to those of skill in the art. For example, acceptable carriers include, but are not limited to, sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions, alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes, paraffins, silicones, talc, silicylate, etc.

Each acceptable carrier used in a pharmaceutical composition or nutritional supplement of the invention must be “acceptable” in the sense of being compatible with the other ingredients of the formulation and not injurious to the subject. Carriers suitable for a selected dosage form and intended route of administration are well known in the art, and acceptable carriers for a chosen dosage form and method of administration can be determined using ordinary skill in the art.

The pharmaceutical compositions and nutritional supplements of the invention may, optionally, contain additional ingredients and/or materials commonly used in pharmaceutical compositions and/or nutritional supplements. These ingredients and materials include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxy methyl cellulose and sodium carbonate; (5) solution retarding agents, such as paraffin; (6) absorption accelerators, such as quaternary ammonium compounds; (7) wetting agents, such as cetyl alcohol and glycerol monosterate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monosterate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; (28) vitamins and minerals; (29) proteins that carry therapeutic or nutritional benefits, such as whey protein and other milk-derived proteins; and (30) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

Pharmaceutical compositions and nutritional supplements suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, or a paste. These formulations may be prepared by methods known in the art, e.g., by means of conventional pan-coating, mixing, granulation or lyophilization processes.

Solid dosage forms for oral administration (capsules, tablets, pills, powders, granules and the like) may be prepared by mixing the active ingredient(s) with one or more acceptable carriers and, optionally, one or more fillers, extenders, binders, humectants, disintegrating agents, solution retarding agents, absorption accelerators, wetting agents, absorbents, lubricants, and/or coloring agents. Solid compositions of a similar type may be employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as capsules, pills and granules, may optionally be scored or prepared with coatings and shells, such as enteric coatings and other coatings well known in the art. The tablets, and other solid dosage forms, may also be formulated so as to provide slow or controlled release of the active ingredient therein. They may be sterilized by, for example, filtration through a bacteria-retaining filter. These compositions may also optionally contain opacifying agents that release the active ingredient only, or preferentially, in a certain portion of the gastrointestinal tract, optionally, in a delayed manner. The active ingredient can also be in a microencapsulated form.

Liquid dosage forms for oral administration include acceptable emulsions, microemulsions, solutions, suspensions, syrups, and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

### EXAMPLES

The following provides several examples, which represent specific and preferred embodiments, of the present invention. In addition to specifying a particular type of chelate that may be used in accordance with the present invention, the following describes the key benefits provided by such compositions (and how such compositions may be used to impart a therapeutic, nutritional, or other type of benefit to a subject).

### Example 1

Vanadium/vanadyl chelated to creatine to support anabolic activity in the building of lean muscle mass, strength and power, fast twitch and slow twitch myofibril power and strength.

### Example 2

Vanadium/vanadyl chelated to arginine to induce vasodilation and promote peripheral blood flow to support healthy blood pressure, optimum growth hormone output, maximum protein synthesis for recovery, optimal workout results, and improved male erectile performance.

### Example 3

Vanadium/vanadyl chelated to citrulline to induce vasodilation and promote peripheral blood flow to support healthy blood pressure, optimum growth hormone output, maximum protein synthesis for recovery, optimal workout results, and improved male erectile performance.

### Example 4

Vanadium/vanadyl chelated to taurine to support insulin activity in the treatment of Type I and Type II diabetes and the optimization of insulin efficiency in the case of subclinical insulin resistance.

### Example 5

Vanadium/vanadyl chelated to phenylalanine to enhance serotonin survival, improve emotional state and weight management, and optimize insulin efficiency to improve fat loss, lean body mass status and healthy weight management.

### Example 6

Vanadium/vanadyl chelated to glutamine to support immune system efficiency and lean muscle anabolism.

### Example 7

Vanadium/vanadyl chelated to glutathione to support immune system efficiency in the course of treatment of disease and disease management, for antimicrobial and anti-aging activity, and for the preservation of tissue structure and function in response to toxicity, poisons and general oxidation pressures.

### Example 8

Vanadium/vanadyl chelated to leucine to support insulin efficiency and anabolic activity in the building of lean muscle mass, strength and power, to support healthy bone mass and reduced body fat, to improve lean body mass to fat body mass proportions in the course of treating obesity, to prevent the effects of osteoporosis and premature aging, to enhance performance in sports, and to support recovery from disease (such as insulin resistance, inflammatory disease or injury such as burns and tissue tears).

### Example 9

A composition that comprises various combinations of the foregoing chelates.

There are many benefits provided by the present invention. For example, the invention provides that vanadium salts, e.g., vanadyl sulfate, may cause gastrointestinal distress at doses required for pharmacological or nutritional effects. In addition, this metallo-mineral readily oxidizes, or can quickly become reduced at certain pH levels (which may contribute to pro-oxidative activity). The invention provides that by tethering the metal to an amino acid ligand, particularly those described herein, a composition is achieved that exhibits enhanced stability, tolerability and bioavailability.

In addition, vanadium salts have been observed to induce nausea when consumed in the doses that are necessary to achieve pharmacological or nutritional results (1.0 mg/pound of body weight, per day). The inventors have found that the novel vanadium and vanadyl amino acid chelates described herein exhibit a substantially improved level of tolerability, bioavailability, and health endpoint achievement in both canine and human subjects. With respect to the bioavailability advantages of the compositions described herein, the invention leverages the prioritized manner in which protein and amino acids are utilized by mammalian digestive systems (vis-à-vis priority absorption by intestinal cells through active- and facilitated passive-absorption), which serves to also impart such preferred absorption and bioavailability to the vanadium/vanadyl metal that is chelated to such amino acids. In addition, as mentioned above, the invention provides that the vanadium and vanadyl amino acid chelates described herein have been found to exhibit an enhanced ability to combat oxidation, thereby preserving and prolonging the functional state of the vanadium and/or vanadyl component thereof.

The recommended daily allowance (RDA) for vanadium is 10-100 mcg. The invention provides that the vanadium chelates described herein can be safely and comfortably administered to a subject, in order to easily satisfy such target doses, in both capsule and powder forms. The invention provides that the effective dose of the chelates described herein is at least 50 mg daily of vanadyl and, more preferably, at least 100 mg daily.

Notwithstanding the foregoing, the invention provides that the dosage amount will vary with the route of administration, the rate of excretion, the duration of the treatment, the identity of any other agents being administered, the age, size, and species of mammal to be treated, and like factors well known in the arts of medicine and nutrition. In general, an effective dose of the vanadium and vanadyl amino acid chelates described herein will be that amount that is the lowest dose effective to produce the desired effect. In addition, the invention provides that the effective dose of the vanadium and vanadyl amino acid chelates may be administered as one, two, three, four, five, six or more sub-doses, administered separately at appropriate intervals throughout the day.

The many aspects and benefits of the invention are apparent from the detailed description, and thus, it is intended for the following claims to cover all such aspects and benefits of the invention which fall within the scope and spirit of the invention. In addition, because numerous modifications and variations will be obvious and readily occur to those skilled in the art, the claims should not be construed to limit the invention to the exact construction and operation illustrated and described herein. Accordingly, all suitable modifications and equivalents should be understood to fall within the scope of the invention as claimed herein.

What is claimed is:

1. A composition comprising vanadium or vanadyl chelated to an amino acid, wherein the amino acid is selected from the group consisting of creatine, arginine, citrulline, taurine, phenylalanine, glutamine, glutathione, and leucine.

2. The composition of claim 1, which further comprises vanadyl sulfate and one or more unbound amino acids selected from the group consisting of creatine, arginine, citrulline, taurine, phenylalanine, glutamine, glutathione, and leucine.

3. The composition of claim 1, which comprises a combination of vanadium or vanadyl chelates, wherein said combination comprises:
(a) vanadium or vanadyl chelated to a first amino acid; and
(b) vanadium or vanadyl chelated to a second amino acid, wherein the first and second amino acid is selected from the group consisting of creatine, arginine, citrulline, taurine, phenylalanine, glutamine, glutathione, and leucine.

4. The composition of claim 3, which further comprises vanadyl sulfate and one or more unbound amino acids selected from the group consisting of creatine, arginine, citrulline, taurine, phenylalanine, glutamine, glutathione, and leucine.

5. The composition of claim 2, which further comprises one or more proteins, amino acids, vitamins, minerals, or combinations thereof.

6. The composition of claim 4, which further comprises one or more proteins, amino acids, vitamins, minerals, or combinations thereof.

7. The composition of claim 1, which comprises vanadium or vanadyl chelated to creatine.

8. The composition of claim 1, which comprises vanadium or vanadyl chelated to arginine.

9. The composition of claim 1, which comprises vanadium or vanadyl chelated to citrulline.

10. The composition of claim 1, which comprises vanadium or vanadyl chelated to taurine.

11. The composition of claim 1, which comprises vanadium or vanadyl chelated to phenylalanine.

12. The composition of claim 1, which comprises vanadium or vanadyl chelated to glutamine.

13. The composition of claim 1, which comprises vanadium or vanadyl chelated to glutathione.

14. The composition of claim 1, which comprises vanadium or vanadyl chelated to leucine.

15. A composition comprising vanadium blended with an amino acid selected from the group consisting of creatine, arginine, citrulline, taurine, phenylalanine, glutamine, glutathione, and leucine.
